(19) United States
(12) Patent Application Publication
(10) Pub. No.: US 2008/0112937 A1
(43) Pub. Date: May 15, 2008

(54) Method of Producing Autologous Embryonic Stem Cells

(76) Inventors: Lewis T Williams, San Francisco, CA (US); Hongbing Zhang, San Francisco, CA (US); Srinivas Kothakota, San Francisco, CA (US); Kristen Pierce, San Francisco, CA (US); Pierre Alvaro Beaurang, San Francisco, CA (US); Keting Chu, Hills Borough, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/632,860

(22) PCT Filed: Jul. 19, 2005
(86) PCT No.: PCT/US05/25580

§ 371 Date: Oct. 15, 2007

Related U.S. Application Data
(60) Provisional application No. 60/590,797, filed on Jul. 22, 2004.

(51) Int. Cl. A61K 48/00; C12N 5/08; C12N 15/09

(52) U.S. Cl. 424/93.21; 435/455; 435/325; 435/366; 435/368; 435/372

(57) ABSTRACT

Aspects of the present invention relate to compositions and methods of reprogramming a somatic cell to give rise to an autologous embryonic stem cell. These methods involve providing a somatic cell of a donor subject, introducing the somatic cell into an embryo of a recipient subject to produce a chimeric embryo, allowing the chimeric embryo to develop further wherein the somatic cell will reprogram, and then selecting an autologous embryonic stem cell that has developed from the somatic cell. The methods and composition of producing pluripotent embryonic stem cells from a donor's own somatic cells invite the possibility of a number of therapeutic applications, including organ transplant and treatment of autoimmune diseases, cancer, and degenerative disorders such as diabetes, Alzheimer's, and Parkinson's.

### PRIORITY CLAIM

This application claims the benefit of provisional application 60/590,797, filed in the United States Patent and Trademark Office on Jul. 22, 2004, the disclosure of which is hereby incorporated by reference.

### TECHNICAL FIELD

The invention relates to the field of embryonic stem cells, specifically to compositions relating to and methods of reprogramming a somatic cell to give rise to pluripotent autologous embryonic stem cells.

### BACKGROUND OF THE INVENTION

A stem cell is a pluripotent or multipotent cell with abilities to self-renew, to remain undifferentiated, and to become differentiated. A stem cell can divide without limit, for at least the lifetime of the animal in which it naturally resides. A stem cell is not terminally differentiated, it is not at the end of a differentiation pathway. When a stem cell divides, each daughter cell can either remain a stem cell or embark on a course that leads to terminal differentiation.

For purposes of tissue engineering, stem cells theoretically provide an inexhaustible supply of cells that, depending on the type, can give rise to some or all of the tissues and organs of an organism. A focus of current research is the promotion of stem cell differentiation to the required lineage, derivation of highly purified cell populations void of carcinogenic potential, and implantation in a form that will replace, or enhance, the function of diseased or degenerating tissues (Odorico J S, et al., Stem Cells (2001) 19:193-204 and Bianco P., et al., Nature (2001) 414:118-121).

An initial step is to select an appropriate stem cell to form the tissue of interest. It is widely known that adult stem cells exist in various tissue niches, including bone marrow, brain, liver, skin, and peripheral blood. While these adult stem cells were originally thought to exhibit only multilineage potential, recent studies have since demonstrated these cells actually exhibit a considerable degree of plasticity and have reported that adult stem cells have the potential to differentiate into a wider range of lineages than previously thought (Krause D S, et al., Cell (2001) 105(3): 369-377), supporting the possibility of autologous tissue generation and transplantation.

While in theory, adult stem cells could be harvested from an individual, incorporated into a tissue construct, and then re-introduced back into the same individual when repair becomes necessary, thereby circumventing the need for immunosuppression, in practice, this presents considerable challenges. Adult stem cells are not easily accessible; they exist at low frequencies, for example, one stem cell per 100,000 bone marrow cells; and they exhibit restricted differentiation potential and poor growth. Collectively, these properties of adult stem cells limit their applicability to tissue engineering.

In contrast to adult stem cells, embryonic stem cells are pluripotent and therefore highly suitable for generating specific cell lineages in vitro. In order to inhibit differentiation, murine embryonic stem cells must be placed in media containing leukemia inhibitory factor or, alternatively, are typically cultured on fibroblast feeder layers. Upon withdrawal of either the leukemia inhibitory factor or feeder cells, most types of embryonic stem cells differentiate spontaneously to form embryoid bodies, comprising derivatives of each of three germ layers.

Continued in vitro culture of murine embryonic stem cells leads to the formation of a range of differentiated cell types, including cardiomyocytes, hematopoietic cells, endothelial cells, skeletal muscle cells, nerve cells, chondrocytes, liver cells, adipocytes, and pancreatic islet cells. This is done by culturing embryonic stem cells under conditions that preferentially encourage differentiation towards a specific lineage of choice, such as providing specific local influences. In most cases, these culture conditions, though potent, still fail to yield a homogeneous population. Accordingly, cells of interest are frequently selected by other means, after differentiation.

Human embryonic stem cells are produced by developing cleavage stage embryos into blastocysts, and removing the inner cell mass (ICM), to retrieve embryonic stem cells. Embryos from which the initial human embryonic stem cell lines are derived can be produced by in vitro fertilization and donated with the informed consent of donor parents. Alternatively, human embryonic stem cells can be derived by a process known as somatic cell nuclear transfer, i.e., cloning. This procedure involves the transfer of the nuclear content of a somatic cell into an enucleated oocyte, either by fusion or by microinjection (McGrath J and Solter D, Science (1983) 229:1300-1302; Campbell K H S, et al., Nature (1996) 380:64-66; Wilmut I, et al., Nature (1997) 385:810-813). The cells are then allowed to undergo embryonic development to the blastocyst stage prior to the isolation from the ICM of embryonic stem cells that will be genetically matched to the tissues of the donor of the nucleus. Embryonic stem cells produced this way are pluripotent and may give rise to a multitude of cells of various lineages when so prompted. Thus, the nuclear content of the somatic cell is reprogrammed during this process to adopt the pluripotency of an embryonic stem cell.

The ability of pluripotent embryonic stem cells to differentiate and give rise to a plurality of specialized mature cells reveals the potential application of these cells as a means to replace, restore, or complement damaged or diseased cells, tissues, and organs. They can be used to prevent and treat autoimmune diseases; cancer; and degenerative diseases, such as diabetes, Alzheimer's and Parkinson's. There are, however, two significant and related limitations of the present art. First, because embryonic stem cells do not exist in adults, they are not readily obtained, particularly from the individual requiring such treatment. Moreover, adult stem cells, as an alternative to embryonic stem cells, are inadequate for these purposes because they are present only at low frequencies and exhibit restricted differentiation potential and poor growth. Second, the immune system, designed to eliminate any cell, tissue, or organ of foreign origin, rejects heterologous transplants, rendering it impractical at best, and most likely impossible, to establish stock human embryonic stem cells. Moreover, while the use of somatic cell nuclear transfer has been shown to adequately reprogram somatic cell nuclear content to adopt pluripotency, the use of somatic cell nuclear transfer has also been debunked as a means to confer total immunocompatability of tissue engineered from embryonic stem cells (Lanza R P, et al., Nature Biotechnol. (1999) 17:1171-1174 and Solter D; Gearhart J, Science (1999) 283: 1468-1470). Therefore, the present art does not provide a feasible or adequate method wherein embryonic stem cells may be produced by reprogramming a somatic cell.

### DISCLOSURE OF THE INVENTION

### SUMMARY OF THE INVENTION

The invention provides a method of producing an autologous embryonic stem cell for a donor subject comprising providing a somatic cell of a donor subject, introducing the somatic cell into an embryo of a recipient subject to produce a chimeric embryo, allowing the chimeric embryo to develop further, and selecting an autologous embryonic stem cell that has developed from the somatic cell. In an embodiment, the somatic cell is introduced into the embryo at or near the eight cell stage. In an embodiment, the autologous embryonic stem cell can be selected from the blastocyst stage of the chimeric embryo.

The somatic cell may be genetically distinguishable from the recipient cell, and may comprise a detectable marker, for example, a reporter gene, such as luciferase, green fluorescent protein, or beta-galactosidase. The somatic cell can be an adult stem cell, for example, an adult mesenchymal, hematopoietic, or neural stem cell. It may be manipulated genetically, for example, to comprise at least one heterologous gene, prior to its introduction into the embryo.

The genetically manipulated heterologous gene may complement a deficiency, for example a recessive chromosomal deficiency, of the somatic cell. It may enhance at least one function or activity of the somatic cell. Examples of heterologous genes suitable for placement in the somatic cell are genes encoding telomerase reverse transcriptase, including human telomerase reverse transcriptase; growth hormone; and phenylalanine hydroxylase. The genetically manipulated heterologous gene may complement sickle cell disease, cystic fibrosis, phenylketonuria, thalassemia, Tay Sachs disease, Fanconi's anemia, Hartnup's disease, pyruvate dehydrogenase deficiency, congenital fructose intolerance (aldolase B deficiency), and/or galactosemia.

The invention also provides a method of reprogramming a somatic cell by providing a somatic cell of a first subject, providing an embryo of a second subject, introducing the somatic cell into the embryo to produce a chimeric embryo, allowing the embryo to develop further, and selecting an embryonic stem cell that is derived from the somatic cell. This method can be performed by introducing the somatic cell into the embryo at or near the eight cell stage. In an embodiment, the first subject is the same as the second subject. The invention further provides an autologous stem cell produced by any of the above-described methods, as well as the progeny of such a cell and a differentiated cell derived from such a cell.

### Definitions

The terms used herein have their ordinary meanings, as set forth below, and can be further understood in the context of the specification.

The term “autologous” is used to describe anything that is derived from an organism's own tissues, cells, or DNA. For example, “autologous transplant” refers to the transplant of tissue or organs derived from the same individual organism. Such procedures are advantageous because they overcome the immunological barrier which otherwise results in rejection.

The term “heterologous” is used to describe something consisting of multiple different elements. As an example, the transfer of one individual's bone marrow into a different individual constitutes a heterologous transplant. A heterologous gene is a gene derived from a source other than the organism.

“Somatic cell” refers to any and all cells that are not germ cells, or gametes. For purposes of this disclosure, a somatic cell is meant to include differentiated cells as well as stem cells, for example adult stem cells, and other cells embraced by the definition accepted by those in the art.

A “stem cell” is a pluripotent or multipotent cell with the ability to self-renew, to remain undifferentiated, and to become differentiated. A stem cell can divide without limit, for at least the lifetime of the animal in which it naturally resides. A stem cell is not terminally differentiated; it is not at the end stage of a differentiation pathway. When a stem cell divides, each daughter cell can either remain a stem cell or embark on a course that leads toward terminal differentiation.

An “embryonic stem cell” is a stem cell that is present in or isolated from an embryo. It can be pluripotent, having the capacity to differentiate into each and every cell present in the organism, or multipotent, with the ability to differentiate into more than one cell type. Embryonic stem cells derived from the inner cell mass of the embryo can act as pluripotent cells when placed into host blastocysts.

An “embryo” is an organism in its early stages of development. It includes a fertilized egg that has begun the process of cell division. At the beginning of this period, the embryo is a totipotent zygote that gives rise to the differentiated cells found in the organism.

An adult stem cell, also called a somatic stem cell, is a stem cell found in an adult. An adult stem cell is found in a differentiated tissue, can renew itself, and can differentiate, with some limitations, to yield specialized cell types of its tissue of origin. Examples include mesenchymal stem cells, hematopoietic stem cells, and neural stem cells.

A “mesenchymal stem cell” (MSC) is an adult pluripotent stem cell progenitor, for example, a blast cell, of one or more mesenchymal lineage, including bone, cartilage, muscle, fat tissue, marrow stroma, and astrocytes. Mesenchyme is embryonic tissue of mesodermal origin, i.e., tissue that derives from the middle of three germ layers. The mesenchyme is populated by mesenchymal cells, which are typically stellate or fusiform in shape. The embryonic mesoderm gives rise to the musculoskeletal, blood, vascular, and urogenital systems, as well as connective tissue, for example, the dermis. Mesenchymal stem cells can be found in, for example, bone marrow, blood, dermis, and periosteum. They may differentiate into, for example, adipose, osseous, stromal, cartilaginous, elastic, and fibrous connective tissues. Their differentiation pathway, for example, whether they become osteoblasts or chondrocytes, may depend on the identity of the agent(s) to which they are exposed.

“Hematopoietic stem cells” (HSCs) are formative pluripotential blast cells found in bone marrow and peripheral blood capable of differentiating into any of the specific types of hematopoietic, or blood, cells such as erythrocytes, lymphocytes, macrophages, and megakaryocytes. A hematopoeitic cell is a cell involved in hematopoeisis, which is the process of forming mature blood cells from precursor cells. In the human adult, hematopoeisis takes place in the bone marrow. Earlier in development, hematopoeisis takes place at different sites during different stages of development; primitive blood cells arise in the yolk sac, and later, blood cells are formed in the liver, spleen, and bone marrow. Hematopoeisis undergoes complex regulation, including regulation by hormones, for example, erythropoietin; growth factors, for example, colony stimulating factors; and cytokines, for example, interleukins.

“Neural stem cells” are stem cells found in adult neural tissue which can give rise to cells that comprise the central nervous system, namely, neurons, astrocytes, and oligodendrocytes. The expression of nestin, a large intermediate filament protein, is characteristic of neural stem cells. In contrast to the results of earlier studies, more recent studies indicate that the adult human brain contains a renewable source of neural stem cells which can be successfully isolated through surgical techniques and expanded in vitro. This capability invites the possibility of autologous transplantation of neural stem cells to treat brain trauma patients, as well as patients with neurodegenerative disorders, such as Parkinson's or Alzheimer's.

A “blastocyst” is an embryo at an early stage of development in which the fertilized ovum has undergone cleavage, and a spherical layer of cells surrounding a fluid-filled cavity is forming, or has formed. This spherical layer of cells is the trophectoderm. Inside the trophectoderm is a cluster of cells termed the inner cell mass (ICM). The trophectoderm is the precursor of the placenta, and the ICM is the precursor of the embryo. Pluripotent embryonic stem cells can be obtained from the ICM of a blastocyst.

The term “chimeric” refers to any union of entities that are derived from different origins. “Chimeric” can indicate an organism composed of at least two types of cells, which may or may not be genetically distinct. For example, a chimeric embryo is formed when a heterologous cell is introduced into an embryo. In this example, both the donor cell and recipient cells are derived from distinct origins. Other examples of a chimeric organism include an organism formed by the fusion of two early blastula stage embryos, by the reconstitution of the bone marrow in an irradiated recipient, or by somatic segregation.

A “gene,” for the purposes of the present disclosure, includes a region of nucleic acid encoding a gene product, as well as all DNA regions which regulate the production of the gene product, whether or not such regulatory sequences are adjacent to the coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

A “reporter gene” typically encodes a gene product which can be easily assayed and which is operably linked to the upstream sequence of another gene and transfected into cells. The assay detects and/or measures a readout signal. A reporter gene can be used to determine which processes are active in the cell type in which it resides, and to determine the effects of test agents on response elements of a gene of interest. Reporter genes are typically downstream of the cloning site of a vector. The reporter gene is typically chosen to be a protein not found in humans and simple to assay for a readout signal. Reporter genes of the invention include, but are not limited to, those commonly used to examine the control of eukaryotic gene expression. One is beta-galactosidase, the product of the lacZ gene, which encodes an enzyme that hydrolyzes the beta galactoside linkage in lactose to yield glucose and galactose. It also hydrolyzes the chromogenic substrate isopropylthiogalactoside (IPTG). Another common reporter gene, luciferase, encodes a gene product that catalyses the reaction between luciferin and ATP, which produces photons of light detectable in a chemiluminescent bioassay for ATP. Alkaline phosphatase catalyzes the cleavage of inorganic phosphate non-specifically from a wide variety of phosphate esters, with a pH optimum greater than about 8. Green fluorescent protein (GFP), a jellyfish protein that fluoresces with green visible light when excited with ultraviolet light, is another commonly used reporter gene.

A “detectable marker” is any marker that is noticeable for the purposes of identifying or distinguishing the presence of something of interest. Detectable markers are typically used to determine the presence of specific cell types within a heterogeneous population of cells. Superior candidates for detectable markers are both easily detected and uniquely expressed on the cell or cells of interest. A detectable marker commonly relied upon is a cell-specific polypeptide expressed on the surface of a cell. Detectable markers may also include any number of other possibilities such as intracellular polypeptides; DNA arrangements (for example the immunoglobulin and T cell receptor loci of B cells and T cells, respectively); molecules such as RNA or lipids; characteristics such as morphology or size; function, such as phagocytosis; or localization. Markers may be detected by a multitude of means, depending largely upon the characteristics of the marker of interest. Detection means may include antibody binding, nucleic acid hybridization enzymatic activity, visual means such as microscopy, staining, fractionation, and functional assays.

In the context of transferring biological material, the term “donor” is used to denote that which is used as a source of the biological material, such as nucleic acid, polypeptides, cells, tissues, or organs. The term “recipient” refers to that organism which accepts the donor biological material. In autologous transfers, the donor and recipient are one and the same, i.e., syngeneic.

A “subject” is an organism from which donor or recipient cells may derived. Species of subjects include, but are not limited to, mouse and human.

In development, a “progenitor cell” is a parent cell committed to give rise to a distinct cell lineage by a series of cell divisions. Specific progenitor cell types may sometimes be identified by markers. For example, hematopoietic progenitor cells bear the marker CD34 on their surface.

The term “precursor cell” refers to a cell from which another cell is formed. It broadly encompasses any cell type that precedes the existence of a later, more mature cell. In contrast to the maturation of progenitor cells, which is marked by cell division, the maturation of precursor cells may include any number of processes or events, including, but not limited to, differential gene expression, or change in size, morphology, or localization site.

A “differentiated cell” is a mature cell that has undergone progressive developmental changes to a more specialized form or function. Cell differentiation is the process a cell undergoes as it matures to an overtly specialized cell type. Differentiated cells have distinct characteristics, perform specific functions, and are less likely to divide than their less differentiated counterparts. An “undifferentiated” cell, for example, an immature, embryonic, or primitive cell, typically has a non-specific appearance, may perform multiple, non-specific activities, and may perform poorly, if at all, in functions typically performed by differentiated cells.

“Progeny” are those born of or derived from another. Progeny include all descendents of the first, second, and all subsequent generations. The term also encompasses those taken, received, or obtained from a parent cell or organism.

Current tools of molecular biology enable scientists to “genetically manipulate” cells of an assortment of organisms in a multiplicity of ways. Bacterial restriction enzymes can manipulate endogenous cellular DNA by addition or subtraction, or by changing the native sequence base by base. Gene manipulation includes techniques known in the art to regulate gene expression by regulating the content of the gene produce. For example, coding regions of DNA may be modified so as to induce cellular expression of truncated proteins, fusion proteins, proteins with other mutations, or wild type proteins to correct existing mutations. Whole organisms may be manipulated genetically, as exemplified by transgenic mice and knockout mice. Such technology provides a premise for human gene therapy.

“Deficiency” indicates the quality or state of having an amount or quality that is lacking or inadequate. In one context, a cell can be considered deficient because it fails to express or expresses inadequate quantities of a given gene product. A deficiency of this nature may adversely affect the cell's ability to function properly. The source of the deficiency may be genetic if the endogenous DNA of the cell is insufficient for the production of a given gene, for example, due to a mutation in the sequence or locus of the gene. Moreover, the nature of the inadequacy may be such that DNA containing a heterologous gene can be introduced so as to overcome the deficiency.

A “function or activity” of a somatic cell refers to any structural, regulatory, or biochemical function of the cell, including any function related to or associated with a metabolic or physiological process. For example, a cell demonstrates activity when it participates in a molecular interaction, when it has therapeutic value in alleviating a disease condition, when it has prophylactic value in inducting an immune response, and when it has diagnostic value in determining the presence of a molecule.

“Telomerase” is a DNA polymerase enzyme that selectively elongates DNA from the telomere, i.e., the end of a chromosome. Telomerases can influence cell aging and play a role in cellular cancer biology. Reverse transcriptases are enzymes that make double stranded DNA copies from single stranded nucleic acid template molecules. Reverse transcriptase plays a role in the replication of some viruses, for example, retroviruses. It is also a standard scientific research tool in the field of molecular biology. The reverse transcriptase polymerase chain reaction (RT-PCR) amplifies specific DNA sequences rapidly, and in vitro. An example of a reverse transcriptase is “human telomerase reverse transcriptase,” a general tumor marker with a reverse transcriptase catalytic subunit (Kirkpatrick K L and Mokbel K, Eur. J. Surg. Oncol. (2001) 27(8):754-760).

Chromosomes are self-replicating cellular DNA that bear, in their nucleotide sequence, the linear gene array. Prokaryotic genomes comprise a single circular chromosome and eukaryotic genomes comprise a number of chromosomes; 23 pairs in a normal human. A “chromosomal deficiency” occurs when there is an error in part or all of a particular chromosome. These errors arise most frequently during mitosis or meiosis and include chromosome loss and mutation. Consequently, genes that ordinarily would have been expressed if the chromosome or portions thereof had remained intact, may fail to be expressed, or may be expressed improperly.

“Recessive” refers to either an allele, mutation, or trait that is phenotypically expressed only when present in a homozygous form or when its missing. In the heterozygous form, the recessive phenotype is masked by the expression of dominant alleles. Whereas heterozygotes do not phenotypically express a recessive gene, they are carriers and may pass the recessive gene to their offspring.

“Reprogramming” a somatic cell means that the differentiated somatic cell gains multipotency or totipotency. It can include the reactivation of genes inactivated during differentiation. The nuclear content of a fully differentiated somatic cell can reprogram inside an enucleated oocyte and give rise to cells of multiple lineages and whole organisms (Campbell K H S, et al., Nature (1996) 39:64-66; Wilmut I, et al., Nature (1997) 385:810-813; U.S. Pat. No. 6,147,276; U.S. Pat. No. 6,252,133; U.S. Pat. No. 6,525,243).

### Methods of Producing a Autologous Embryonic Stem Cells

One aspect of the invention provides a method of producing an autologous embryonic stem cell for a donor subject, which involves providing a somatic cell from the donor subject, introducing it into an embryo of a recipient subject to produce a chimeric embryo, allowing the chimeric embryo to further develop, and then selecting an autologous embryonic stem cell that has developed from the somatic cell.

### Introduction of Somatic Cells

As discussed above, an embryonic stem cell is pluripotent, or multipotent. It may have the ability to differentiate into one or more cell types of an organism. Accordingly, such a cell is potentially useful to replace, restore, or complement damaged or diseased cells, tissues, and organs. In an embodiment, the invention provides a method of producing an autologous embryonic stem cell from a somatic stem cell of a donor subject. Subsequent re-introduction of the embryonic stem cell into the donor subject overcomes the potential problem of rejection, since the embryonic stem cell is autologous and is returning to the host from which it was derived. The individual suffering from the disease provides somatic cells, which give rise to embryonic stem cells that may then be returned to that same individual to restore, replace, or complement defective cells, tissues, and organs.

This aspect of the invention initially requires somatic cells. Suitable somatic cell include all cells of an organism, with the exception of germ cells (gametes). Somatic cells are typically differentiated and fully mature, and have specific and distinctive functions. T cells, for example are localized to lymphoid organs and peripheral blood, where they alert the organism surveying the body for the presence of pathogens or component parts of pathogens. Keratinocytes, by comparison, coalesce amongst one another forming a multi-layered physical barrier against the outside world. Moreover, somatic cells may also include undifferentiated non-germ cells that are involved in development, for example, adult stem cells. Adult stem cells, or somatic stem cells, exist in differentiated tissue of adult beings.

Sources of somatic cells are numerous and well known in the art. Numerous types of human cell lines, for example, are readily available commercially, and are commonly used for experimental purposes. Additionally, such cells may be derived directly from human sources by collecting biological samples. Somatic hematopoietic cells, for example, may be obtained easily by conventional blood drawing or by biopsy.

In an aspect of the invention, somatic cells are introduced into an embryo. Upon fertilization, the zygote undergoes cleavage and rapid cell division, as it passes down the oviduct and into the uterus. In mammalian development, cleavage is holoblastic, that is the cleavage furrow extends through the entire cell, and the cleavage symmetry is rotational. The dividing zygote floats freely, absorbing nutrients from the uterus. After three cleavage events, the early eight cell stage undergoes compaction, wherein the blastomeres huddle together, maximizing their contact with other blastomeres and form a compact ball of cells. Next, the compacted embryo divides to produce a sixteen cell morula. The morula consists of a small group of internal cells surrounded by a larger group of external cells (Barlow P W and Sherman M I, et al., J. Embryol. Exp. Morphol. (1972) 27(2):447-465). Most of the descendants of the external cells become the trophoblast cells, which will produce no embryonic structures and will instead form the chorion, which is the embryonic contribution to the placenta. The embryo is derived primarily from the descendants of the inner cells of the morula. These cells, along with the occasional cell dividing from the trophoblast during the transition to the thirty-two cell stage, generate the inner cell mass (ICM).

A blastocyst is formed when the trophoblast cells secrete fluid into the morula to create a blastocoel, with the ICM positioned on one side of the ring of trophoblast cells. The developing embryo then undergoes gastrulation where the three primary germ layers, endoderm, mesoderm, and ectoderm, are formed, the basic body plan is established, and cellular interactions take place that will result in neurulation and organogenesis.

The invention provides for the somatic cell to be provided to the embryo when the embryo is at or near the blastocyst stage or earlier, for example, at the eight cell stage. In an embodiment, the somatic cell is introduced in the morula stage or the blastocyst stage.

Murine embryos are readily available and their development is well-characterized in the art. Further the conditions under which they ideally grow are in the art. Moreover, embryonic cells derived from other animals are also easily obtained and well known in the art. There are numerous available sources of human embryos as well, for example, fertility clinics.

According to a method of the invention, a somatic cell is introduced to an embryo to form a chimeric embryo. In this aspect, the introduction of a somatic cell to an embryo generally means the coming together of one or more somatic cells with one or more embryonic cells. There are multiple varied ways in which a somatic cell may be introduced to an embryo and the invention provides broadly for any means in which one or more somatic cell comes together with one or more embryonic cells. For example, a somatic cell and an embryonic cell can come together in vitro simply by placing a somatic cell together with an embryo under appropriate tissue culture conditions well known in the art. Moreover, a somatic cell and an embryo can come together in vivo. One or more somatic cells and one or more cells of the embryo, for example, can be transferred concurrently to the uterus of a pseudopregnant mouse, for example, a female mouse mated with a vasectomized male, wherein the stimulus of mating elicited hormonal changes leading to uterine receptivity. Another example in which a somatic cell and an embryo can come together in vivo is by injecting one or more somatic cells into the inner cell mass of a blastocyst and then implanting that chimeric embryo into a pseudopregnant mouse.

The resultant product of the union between a somatic cell and an embryo is a chimeric embryo. This embryo is considered chimeric because it consists of groups of two different cells of diverse origins, thereby reflecting the distinctness of the two different cells.

An aspect of the invention provides for a method where the chimeric embryo, the product of the introduction of a somatic cell to an embryo, is allowed to develop further. Reports of somatic cell nuclear transfer have shown that the nuclear content of a terminally differentiated somatic cell, when allowed to develop further in an enucleated oocyte, can reprogram and acquire the potential to differentiate in the manner of an embryonic stem cell. These studies suggest that development within an embryonic environment, i.e., advancement through the various stages of embryogenesis, facilitates the reprogramming and the acquisition of multipotency or pluripotency. According to the invention, following the introduction of the somatic cell to the embryo, the chimeric embryo will undergo further growth and development, advancing through the various stages of embryogenesis. The cessation of the permitted development can be marked by selection of the autologous embryonic stem cell. It is well known in the art that embryonic stem cells can be isolated from the ICM of the blastocyst. The invention provides that the autologous embryonic stem cell is selected from the chimeric embryo at the blastocyst developmental stage. Accordingly, in an embodiment, the chimeric embryo is allowed to develop further, to a stage where the autologous embryonic stem cell or cells produced are easily selected and isolated, for example, at or around the blastocyst stage of development.

This aspect of the invention further provides that the autologous embryonic stem cell produced is to be selected from the chimeric embryo. An autologous embryonic stem cell of the donor subject is to be selected for example, isolated, from cells of the recipient embryo. Selection of the autologous embryonic stem cell from cells of the recipient embryo involves discerning between these cells. More specifically, the autologous embryonic stem cell is identified to be selected, or the non-embryonic stem cells are otherwise removed.

Multiple ways of distinguishing one or more cells from a heterogenous collection of cells are known in the art. These methods can be broadly classified as either positively identifying and retaining the cell of interest, i.e. the autologous embryonic stem cell, or negatively identifying and then removing or deleting all other cell populations. There are multiple ways to remove the non-autologous embryonic stem cells, leaving the autologous stem cells intact. One such way is to treat the embryo to dislodge the trophectoderm of the embryo or portion thereof. For example, the embryo may be treated by washing with an appropriate blastocyst culture medium, for example G2 or S2 (Scandanavian-2 medium), to dislodge the trophectoderm or a portion thereof, thereby leaving the ICM remaining harboring autologous embryonic stem cells. Alternatively, or additionally, the embryo may be treated with an antibody or antiserum reactive with epitopes on the surface of the trophectoderm. Antibody-bound trophectoderm cells may then be subjected to complement, which dislodges the trophectoderm away from the autologous embryonic stem cell. Yet another way in which to select an autologous embryonic stem cell away from the remaining embryo is to gently homogenize the embryo so as to obtain a single cell suspension. This suspension can then be subject to a selection method. Antibodies directed to surface polypeptides expressed on either the autologous embryonic stem cell or on the remaining cells may provide both positive and negative selection protocols.

In an embodiment, the invention provides a method of genetically manipulating the autologous embryonic stem cell at the level of the precursor somatic cell to render the cell identifiable. The somatic cell, prior to introduction to the embryo, may be transfected with an expression vector encoding an identifiable gene product. Upon introduction of the somatic cell to the embryo, and further development of the chimeric embryo, the somatic cell-derived autologous embryonic stem cell can be identified by the enforced expression of that unique gene product. By way of example, the POU (named after the transcription factors Pit, Onc, and Unc) transcription factor Oct4 is a suitable marker of undifferentiated murine embryonic stem cells. Likewise, the process of differentiation is associated with a reduction in Oct4 expression and activity. Accordingly, a reporter gene containing an Oct4-binding site upstream of a gene for a detectable marker may be introduced heterologously into either the somatic cell prior to its introduction to the embryo or to the autologous embryonic stem cell. The presence of the active Oct4 transcription factor will drive transcription and translation of the detectable marker, thereby signaling the presence of an undifferentiated embryonic stem cell (Pesce M and Schöler H R, Stem Cells (2001) 19(4):271-278). This type of technology is widely used and well known in the art.

Upon selection and enrichment or isolation, the autologous embryonic stem cell may then be characterized and subsequently used for therapeutic applications. In an embodiment, an autologous embryonic stem cell produced by the methods of the invention remains viable. It is not placed in conditions which would induce extra-embryonic differentiation, cell death, or proliferation. To prevent differentiation, the autologous embryonic stem cells may be cultured on a fibroblast feeder layer. The fibroblast feeder can be maintained at a density of approximately 25,000 human or 70,000 murine cells per cm²; it may be established approximately 6-48 hours prior to the addition of the embryonic stem cells. Optionally, feeder cells may be treated to induce cell arrest by methods including, but not limited to, irradiation and exposure to mitomycin C. ICM cells, such as autologous embryonic stem cells, may be cultured on a fibroblast feeder layer and maintained in embryonic stem cell medium. A suitable embryonic stem cell medium is, for example, Dulbecco's Minimum Essential Medium, without sodium pryuvate, with glucose (4500 mg/L), supplemented with 20% fetal bovine serum, beta-mercaptoethanol (0.1 mM), non essential amino acids, glutamine (2 mM), and penicillin (50 μ/ml), and streptomycin (50 μ/ml). As an alternative to co-culturing with a fibroblast feeder layer, embryonic stem cell media containing leukemia inhibitory factor may also be used to prevent differentiation of an autologous embryonic stem cell The methods of culturing stem cells including those derived from the ICM, to inhibit differentiation, cell death, and proliferation are widely practiced and well known to those skilled in the art.

An autologous embryonic stem cell produced by the methods of the invention has broadly ranging therapeutic applications. Numerous human diseases and conditions may benefit from the ability to produce an autologous embryonic stem cell from an adult somatic cell capable of giving rise to any and all cells of the body. Notably, aspects of the invention eliminate many of the drawbacks that accompany tissue and organ transplant strategies. For purposes of transplantation, the invention provides a source of replacement tissues or organs. The recipient is also the donor; this overcomes the problem of immune rejection, thereby precluding a requirement for immunosuppression. The technology provided by this aspect also allows for the possibility of regenerating organs harboring tumors. Current cancer therapies such as chemotherapy and radiation treatment are inadequate in that both cancerous and non-cancerous cells alike are often eliminated. This aspect of the invention provides a means to regenerate cells lost to cancer therapy. Therefore, these aspects of the invention alleviate the non-specific killing associated with present cancer therapies.

An aspect of the invention provides for a method of producing an autologous stem cell, as above, by introducing a somatic cell into a recipient embryo, wherein the somatic cell is genetically distinguishable from the recipient cell. Specifically, this aspect provides that the somatic donor and recipient embryo cells are distinguished because their genetic content is not identical. This distinction may arise because the somatic cell and embryo cells are derived from different sources, and/or because the two cell populations are deliberately made to be genetically distinguishable. The first condition can arise naturally when two cell populations from non-identical sources, i.e. not from identical offspring, inbred strains, or clones. Conversely, the second condition arises artificially. Standard tools of molecular biology can be used to exogenously add or delete nucleic acid fragments, or to otherwise modify, for example introduce mutations, into the genome of a cell. Accordingly, scientists are able to regulate gene expression in a multitude of ways. One such example is provided by an aspect of the invention in which the donor and recipient cells are genetically distinguishable due to expression of detectable markers. For example, nucleic acid sequences encoding detectable markers can be exclusively transfected into the somatic cell. There are many such detectable marker systems commercially available and well known in the art.

One example of a detectable marker is a selectable system, whereby an exogenous gene is transfected into a cell encoding a polypeptide that confers resistance against an otherwise toxic agent. Subsequent exposure to that toxic agent will kill untransfected cells, but not affect transfected cells harboring a transgene that confers protection. A number of selectable systems may be used, including, but not limited to, the herpes simplex virus thymidine kinase, hypoxanthine-guanine phosphoribosyltransferase, and adenine phosphoribosyltransferase genes. These can be employed in tk⁻, hgprt⁻ or aprt⁻ cells, respectively. Also, antimetabolite resistance can be used as the basis of selection for dihydrofolate reductase, which confers resistance to methotrexate; xanthine/guanine phosphoribosyl transferase, which confers resistance to mycophenolic acid; neomycin, which confers resistance to the aminoglycoside G-418; and hygromycin, which confers resistance to hygromycin genes. Additional selectable genes have been described, such as trpB, which allows cells to utilize indole in place of tryptophan; hisD, which allows cells to utilize histinol in place of histidine; and ornithine decarboxylase, which confers resistance to the ornithine decarboxylase inhibitor 2-(difluoromethyl)-DL-ornithine.

Other detectable markers useful herein include cell surface markers such as alkaline phosphatase, nerve growth factor receptor, or any other suitable membrane-associated moiety. Representative examples of such markers and associated prodrug molecules include alkaline phosphatase and various toxic phosphorylated compounds such as phenolmustard phosphate, doxorubicin phosphate, mitomycin phosphate and etoposide phosphate; β-galactosidase and N-[4-(β-D-galactopyranosyl) benyloxycarbonyl]-daunorubicin; azoreductase and azobenzene mustards; β-glucosidase and amygdalin; β-glucuronidase and phenolmustard-glucuronide and epirubicin-glucuronide; carboxypeptidase A and methotrexate-alanine; cytochrome P450 and cyclophosphamide or ifosfamide; DT diaphorase, and 5-(aziridine-1-yl)-2,4,dinitrobenzamide (CB1954) (Cobb L M, et al., Biochem Pharmacol. (1969) 18(6):1519-1527; Knox R J, et al., Cancer Metastasis Rev. (1993) 12(2): 195-212); β-glutamyl transferase and β-glutamyl p-phenylenediamine mustard; nitroreductase and CB1954 or derivatives of 4-nitrobenzyloxycarbonyl; glucose oxidase and glucose; xanthine oxidase and hypoxanthine; and plasmin and peptidyl-p-phenylenediamine-mustard. Nonimmunogenic markers may also be made by expressing an enzyme in a compartment of the cell where it is not normally expressed.

In an embodiment, the donor and recipient cells may be genetically distinguishable by virtue of reporter genes, as an example of detectable markers. This aspect of the invention provides for the use of luciferase, green fluorescent protein, and β-galactosidase as non-limiting examples of reporter genes. Donor and recipient cells can be easily distinguished by the use of these commonly used reporter genes. Nucleic acid sequences encoding a reporter gene can be transfected into the somatic cell of the donor subject prior to the introduction of the somatic cell into the embryo. Because these genes are not typically expressed in human cells, untransfected recipient embryo cells will not harbor those genes and will not express its products. Therefore, the detection of the reporter gene product indicates the presence of the donor somatic cell. This may be useful when the autologous embryonic stem cell is to be selected from the chimeric embryo, as per certain aspects of the invention. Reporter genes can be obtained from commercially available plasmids, using techniques well known in the art (for example Sambrook J., et al, Molecular Cloning, A Laboratory Manual, Cold Spring Harbor Laboratory Press, NY, Vols. 1-3 (1989)).

In an embodiment, the invention provides a method of producing an autologous stem cell by introducing an adult cell into a recipient embryo. Adult stem cells suitable for use in this embodiment include mesenchymal stem cells, hematopoietic stem cells, and neural stem cells.

Mesenchymal stem cells can be found in bone marrow, blood, dermis, and periosteum. They can differentiate into, for example, adipose, osseous, stroma, cartilaginous, elastic, and fibrous connective tissues. Their differentiation pathway, for example, into cells such as osteoblasts and chondrocytes, depends on the agent(s) to which they are exposed. Mesenchymal stem cells are available or may be derived from embryonic stem cells exposed to factors and conditions that drive the differentiation of embryonic stem cells towards the mesenchymal lineage. The method of promoting mesenchymal lineage differentiation is well known in the art.

Methods of obtaining hematopoeitic stem cells are also well known in the art. Hematopoietic stem cells can be obtained, for example, by subjecting low density mononuclear bone marrow cells to counterflow elutriation and then recovering CD34⁺ cells from the fractions containing smaller cells. The stem cells are isolated from bone marrow cells by panning the bone marrow cells with antibodies which bind unwanted cells, such as CD4⁺ and CD8⁺ (T cells), CD45⁺ (pan B cells), GR-1 (granulocytes), and lad (differentiated antigen presenting cells). The expression of a particular antigen or antigens on the cell surface or in the cytoplasm and the intensity of expression can indicate the stage of maturation and lineage commitment of the hematopoietic stem cell. The hematopoietic stem cells may be differentiated in vitro into clinically important immune cell types using cytokines such as, for example, GM-CSF, IFN-γ, and TNF-α.

Neural adult stem cells are available or may be derived from embryonic stem cells exposed to factors and conditions that drive the differentiation of embryonic stem cells towards the neural lineage. The method of promoting neural lineage differentiation is well known in the art.

In an embodiment, the invention provides for a method of producing an autologous stem cell, as above, by introducing a somatic cell into a recipient embryo which is at or near the eight cell stage. Prior to the eight-cell stage, the embryo generally has insufficient space to accommodate a donor cell. It is known in the art that the embryo is advantageously at an early stage prior to introduction of other cells. The invention provides that the recipient embryo may further develop, subsequent to the introduction of the donor cell.

In an embodiment, the chimeric embryo is at a stage at or preceding that of the blastocyst stage and will later enter the blastocyst stage of development and/or proceed beyond the blastocypt stage. The characteristics of the blastocyst stage have been well studied such that one skilled in the art can recognize when that stage has been achieved. Additionally, it is well known in the art that embryonic stem cells can be isolated from the ICM of the blastocyst. Therefore, the blastocyst represents an appropriate developmental stage at which to select an autologous embryonic stem cell that has developed from the somatic cell.

### Expression of Heterologous Genes

An aspect of the invention provides a method of producing an autologous stem cell, by providing a somatic cell which has been manipulated genetically prior to its being introduced into the embryo. Genetic manipulation of a cell, tissue, or organism includes manipulating nucleic acids to affect gene expression, thereby potentially regulating diverse facets of the production of specific gene products. This technology, in effect, grants the ability to induce, cease, enhance, or diminish expression of endogenous or exogenous genes. By way of example, a somatic cell can be transfected with a sequence of nucleic acids, an expression vector that includes both regulatory sequences, for example promoter and coding sequences, which encode a gene product. Upon transfection, the cell will express the exogenous gene product when appropriate, depending on the nature of the promoter.

The DNA sequences encoding the proteins can be obtained from natural sources, such as an organism or tissue sample, for example, or can be synthetically produced using sequences obtained from the literature or from publicly accessible databases. These methods and resources are widely employed and known in the art.

In an embodiment, genetic modification of the stem cells can be performed by transfection using methods known in the art, including CaPO₄ transfection, DEAE-dextran transfection, by protoplast fusion, electroporation, lipofection, and the like. With direct DNA transfection, cells can be modified by, for example, particle bombardment, receptor mediated delivery, and/or cationic liposomes.

The cells can also be genetically manipulated by the introduction of the full-length gene sequences of the proteins. The full-length gene sequences can be isolated from vectors or synthesized completely or in part using various oligonucleotide synthesis techniques known in the art, such as site-directed mutagenesis and polymerase chain reaction (PCR) techniques where appropriate. In particular, one method of obtaining nucleotide sequences encoding the desired sequences is by annealing complementary sets of overlapping synthetic oligonucleotides produced in a conventional, automated polynucleotide synthesizer, followed by ligation with an appropriate DNA ligase and amplification of the ligated nucleotide sequence via PCR (for example, Jayaraman K, et al., Proc. Natl. Acad. Sci. USA (1991) 88:4084-4088). Additionally, oligonucleotide directed synthesis (Jones et al., Nature (1986) 54:75-82), oligonucleotide directed mutagenesis of pre-existing nucleotide regions (Riechmann L, et al., (1988) Nature, 332:323-327; Verhoeyen M, et al., Science (1988) 239:1534-1536), and enzymatic filling-in of gapped oligonucleotides using T₄ DNA polymerase (Queen C, et al. (1989) Proc. Natl. Acad. Sci. USA 86:10029-10033) can be used to provide the sequences.

Once coding sequences have been prepared or isolated, they can be cloned into any suitable vector or replicon. Numerous cloning vectors are known to those of skill in the art, and the selection of an appropriate cloning vector is a matter of choice. Suitable vectors include, but are not limited to, plasmids, phages, transposons, cosmids, chromosomes, and viruses which are capable of replication when associated with the proper control elements.

The coding sequence is then placed under the control of suitable control elements, depending on the system to be used for expression. Thus, the coding sequence can be placed under the control of a promoter, ribosome binding site, and, optionally, an operator, so that the DNA sequence of interest is transcribed into RNA by a suitable transformant. The coding sequence may or may not contain a signal peptide or leader sequence which can later be removed by the host in post-translational processing (for example, U.S. Pat. Nos. 4,431,739; 4,425,437; 4,338,397).

Expression vectors suitable for use in the present invention can be constructed by any conventional method. For example, the expression vector can be constructed such that the gene of interest is located in the vector under the control of the appropriate regulatory sequences. Modification of the sequences encoding the gene of interest may be desirable to achieve this end. For example, in some cases it may be necessary to add to the coding sequence of the gene of interest so that it can be attached to the control sequences in the correct reading frame. The control sequences and other regulatory sequences may be ligated to the coding sequence prior to insertion into a vector. Alternatively, the coding sequence can be cloned directly into an expression vector which already contains the control sequences and an appropriate restriction site. Several possible vector systems are available and known in the art. Some vectors use DNA elements which provide autonomously replicating extra-chromosomal plasmids, generally derived from animal viruses. Other vectors include Vaccinia virus expression vectors. Still other vectors integrate the desired polynucleotide into the host chromosome.

The genetically manipulated cells can be selected by introducing one or more markers, for example an exogenous gene which allows for the selection of cells harboring the expression vector. The selectable marker gene can either be directly linked to the DNA sequences to be expressed, or introduced into the same cell by co-transformation. Additional elements may also be needed for optimal synthesis of mRNA. These elements may include splice signals, as well as transcription termination signals.

In one aspect, DNA encoding the protein of interest can be introduced into the cells by the method of Remy J S, et al., Proc. Natl. Acad. Sci. USA (1995) 92(5):1744-1748, which is a modular transfection system based on lipid-coating the polynucleotides. The particle core is composed of the lipopolyamine-condensed polynucleotide in an electrically neutral ratio to which other synthetic lipids with viral properties are hydrophobically adsorbed. Usually a zwitterionic lipid, such as dioleoyl phosphatidylethanolamine, can be used to coat the nucleotides.

Another targeted delivery system for the polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles which are useful as delivery vehicles in vitro and in vivo. It has been shown that large unilamellar vesicles, which range in size from 0.2-4.0 μm, can encapsulate a substantial percentage of an aqueous buffer containing large macromolecules. RNA, DNA and intact virions can be encapsulated within the aqueous interior and be delivered to mammalian cells, plant, yeast, and bacterial cells (Fraley R, et al., J. Biol. Chem. (1980) 255(21):10431-10435). The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine, and distearoylphosphatidylcholine.

In another aspect of the invention, viral vectors can be used to transfect the cells with the genes encoding the proteins. Viral vectors include retroviruses (including lentiviruses), adenoviruses, adeno-associated viruses and herpes simplex virus type I. Such vectors may additionally require helper cell lines for replication and stem or differentiated cell specific regulatory sequences. Viral vectors that carry a heterologous gene (transgene) generally will contain viral, for example retroviral long terminal repeat (LTR), simian virus 40 (SV40), or cytomegalovirus (CMV); or tissue-specific promotes, for example liver-specific, such as albumin (Connelly S, et al., Hum. Gene Ther. (1995) 6(2):185-193; Milos P M and Zaret K S, Genes Dev. (1992) 6(6):991-1004) or pancreatic cell-specific promoters, such as insulin.

As will be evident to one of skill in the art, the DNA sequence encoding a protein or a fragment of a protein can be targeted to a chosen locus in the stem cell genome. In one aspect of the invention, the locus can be selected such that it has a higher targeting frequency, is not hypo-insufficient, and/or is capable of ubiquitously expressing the inserted DNA at high frequency. The choice of the locus can depend on the source of the stem cell and the method of transfection. For example, if mouse ES cells are selected to be genetically modified using homologous recombination, then the ROSA 26 locus can be targeted for the incorporation of the DNA sequences. Any gene loci can be used in the practice of the aspect of the invention provided targeting one copy of the gene will not result in a haploinsufficient phenotype. Thus, the locus can be chosen from ROSA 26, ROSA 5, ROSA 11, G3BBP(BT5), phosphoglycerate kinase, and actin loci.

One aspect of the invention provides a method of producing an autologous stem cell, as above, by providing a somatic cell which has been manipulated genetically to comprise at least one heterologous gene, prior to its being introduced into the embryo. An exogenous gene, that is, one not derived from the somatic cell, is considered a heterologous gene, for purposes of this aspect of the invention. Therefore, as provided by this aspect, at least one heterologous gene is introduced to the somatic cell that did not formerly exist in the cell.

Cells may exhibit one or more gene defects, for example mutations, such that the expression of the gene product is altered, thereby resulting in a deficiency within the cell. Sometimes this defect occurs in regulatory sequences of the nucleic acid, for example the promoter region, thereby causing improper regulation of the expression of the gene product. Consequences of a defect in the promoter region of a gene include increased or decreased expression, which, depending on the gene product, may have deleterious results, such as disease causation. Likewise, defects may also occur in coding regions, for example exons that encode for the amino acid comprising the gene product, thereby altering the gene product itself. Such a defect may render the cell unable to produce the normal version of its gene product. Instead, the cell may produce an abnormal polypeptide, for example misfolded polypeptide or truncated protein. Therefore, a defect or a mutation in a gene can result in a deficiency in the cell. This, in turn, may lead deleterious results in vivo, such as disease.

A genetic manipulation to comprise at least one heterologous gene, as provided by this aspect of the invention, is practical for complementing a gene or genes. There are numerous conditions, such as those described above, where a defect in a gene may result in a deficiency in that cell, which could cause human disease or another undesirable condition. A deficiency of this nature in the cell can frequently be complemented by genetic manipulation comprising a heterologous gene, thereby restoring the cell to normal function. A normal, non-mutated, heterologous gene can be exogenously introduced into the mutant cell to complement or correct the defect arising from an endogenous mutation. For the purposes of the invention, a somatic cell can be readily obtained as above, and may contain a deficiency. That deficiency may be complemented by the genetic manipulation of a heterologous gene as described above. Methods for using a heterologous gene to complement a cell deficiency are widely known in the art. This approach to correcting genetic defects may be favored compared to other prospects such as regular administration of a polypeptide, for example insulin, which can be more costly and burdensome.

One aspect of the invention provides a method of producing an autologous stem cell, as above, by providing a somatic cell, which has been manipulated genetically to comprise at least one heterologous gene that enhances at least once function or activity of the somatic cell, prior to its introduction into the embryo. In some conditions, a cell is not completely deficient in the function, but may instead have reduced, diminished, enhanced, or abnormal function or activity. This altered function or activity may be attributed to a genetic defect; in such a case, a heterologous gene may enhance the function or activity of this cell.

By way of example, a heterologous gene can increase the production of a gene product in a cell producing some, but still insufficient, amounts of that gene product. A heterologous gene, for purposes of this aspect, may be a sequence of nucleic acid that includes a unique promoter sequence designed so as to be constitutively active. A promoter of this type would initiate and maintain the continued synthesis of its downstream gene product. Such a heterologous gene would be useful for cells that produce insufficient amounts of that gene product, and would thereby enhance its function or activity. Similarly, a heterologous gene, for purposes of this aspect, may be a sequence of nucleic acid that includes a small sequence that encodes only a few peptides. Such a short polypeptide, for example a tag, may, when added to the polypeptide encoded by the coding region of the heterologous gene, confer greater stability of the polypeptide. In this way, the polypeptide produced by the heterologous gene may be more long-lived thereby enhancing the function or activity of the cell that produces insufficient amounts of that polypeptide. These general examples illuminate only some of the many ways in which a heterologous gene may enhance the function or activity of a cell as provided by this aspect of the invention.

One aspect of the invention provides a method of producing an autologous stem cell, by providing a somatic cell, which has been manipulated genetically to comprise at least one heterologous gene that encodes telomerase reverse transcriptase, prior to its being introduced into the embryo. The DNA polymerase enzyme, telomerase, selectively elongates DNA from the telomere, i.e., the end of a chromosome. Telomeric DNA contains multiple, for example, hundreds, of tandem repeats of a hexanucleotide sequence. One strand of telomeric DNA is G-rich at the 3′ end, and slightly longer than the other strand. Telomeric DNA can form large duplex loops, wherein the single-stranded region at the very end of the structure loops back to form a DNA duplex with another part of the repeated sequence, displacing a part of the original telomeric duplex. This loop-like structure is formed and stabilized by specific telomere-binding proteins. These structures protect and mask the end of the chromosome.

The telomeric loop-like structures are generated by telomerase. The telomerase enzyme contains an RNA molecule that serves as the template for elongating the G-rich strand of telomeric DNA. Thus, the enzyme carries the information necessary to generate the telomere sequences. Telomerases also have a protein component, which is related to reverse transcriptases. Telomerases can influence cell aging, and play a role in cellular cancer biology.

Reverse transcriptases are enzymes that make double stranded DNA copies from single stranded nucleic acid template molecules. Typically, a reverse transcriptase is a DNA polymerase that can copy both RNA and DNA templates, and has an integral RNase H activity (Lim D, et al., J. Virol. (2002) 76(16):8360-8373). The two enzymatic domains of reverse transcriptase reflect these two activities; the first is a DNA polymerase domain that can use either RNA or DNA as a template to synthesize either the minus-strand or the plus strand of DNA, and the second is an RNase H domain that degrades the RNA in RNA-DNA hybrids (Wu A M and Gallo R C, CRC Crit. Rev. Biochem. (1975) 3(3):289-347).

Reverse transcriptase plays a role in the replication of some viruses, for example, retroviruses. It copies the retroviral RNA genome to produce a single minus strand of DNA, and then catalyzes the synthesis of a complementary plus strand. Accordingly, reverse transcriptase is a therapeutic target for conditions that involve retroviruses, for example, Acquired Immune Deficiency Syndrome (AIDS). A number of anti-retroviral drugs inhibit reverse transcriptase (Frank I, Clin. Lab. Med. (2002) 22(3):741-757).

Reverse transcriptase is also a standard scientific research tool in the field of molecular biology. The reverse transcriptase polymerase chain reaction (RT-PCR) amplifies specific DNA sequences rapidly, and in vitro. RT-PCR can detect trace amounts of RNA and DNA, and is used in a wide range of applications, including forensics, the diagnosis of genetic diseases, determination of the prognosis of diagnosed diseases, and the detection of viral infection (Alberts, B, et al., Molecular Biology of the Cell, 3rd ed. (1994) Garland Publishing, New York, N.Y.). For example, reverse transcriptase is used to diagnose cancer and to provide prognostic information about the predicted survival of patients with prostate cancer (Kantoff P W, et al., J. Clin. Oncol. (2001) 19(12):3025-3028).

A related aspect of the invention additionally provides a method of producing an autologous stem cell by providing a somatic cell, which has been manipulated genetically to comprise at least one heterologous gene that encodes telomerase reverse transcriptase, specifically human telomerase reverse transcriptase, prior to its being introduced into the embryo. As above, an expression vector may be synthesized to embrace a promoter and coding sequence for a telomerase reverse transcriptase gene or specifically human telomerase reverse transcriptase. This vector can readily be introduced into a somatic cell, which will then produce telomerase reverse transcriptase when appropriate. Moreover, the DNA sequences encoding the telomerase reverse transcriptase, human or otherwise, can be obtained from natural sources, such as an organism or tissue sample, for example, or can be synthetically produced using sequences obtained from the literature or from publicly accessible databases. These methods and resources are widely employed and known in the art.

Yet another aspect of the invention provides a method of producing an autologous stem cell by providing a somatic cell, which has been manipulated genetically to comprise at least one heterologous gene that complements a chromosomal deficiency of the somatic cell, prior to its being introduced into the embryo. Chromosomal deficiencies may result in human disease or other disorder. Turner syndrome is an example of a disease caused by a chromosomal deficiency, occurring when females inherit only one X sex chromosome. Accordingly, individuals with Turner syndrome have a genotype of XO° for the sex chromosome.

An appropriate heterologous gene may complement, in part or in full, the chromosomal deficiency. Accordingly, this aspect provides that a somatic cell may be manipulated genetically to comprise at least one heterologous gene that complements the chromosomal deficiency. This may be accomplished in the same way other genetic manipulations may be accomplished as described above and are well known in the art, for example, by introducing a heterologous gene that encodes regions of the chromosome that are lacking due to the chromosomal deficiency into a somatic cell. This genetic manipulation may complement and overcome the deficiency. Further, the DNA sequences encoding the sequences required for complementation can be obtained from natural sources, such as a normal organism or tissue sample, for example, or can be synthetically produced using sequences obtained from the literature or from publicly accessible databases. These methods and resources are widely employed and known in the art.

Similarly, the invention also provides a method of producing an autologous stem cell, by providing a somatic cell, which has been manipulated genetically to comprise at least one heterologous gene that complements a recessive chromosomal deficiency of the somatic cell, prior to its being introduced into the embryo. If homozygous recessive, a chromosomal deficiency will manifest phenotypically. However, a heterozygous recessive chromosomal deficiency may be compensated for by the genes of the paired homologous chromosome. In such a case, a heterologous gene may complement the recessive chromosomal deficiency. Accordingly, this aspect provides for such complementation, and can be accomplished as mentioned above.

An aspect of the invention provides a method of producing an autologous stem cell by providing a somatic cell, which has been manipulated genetically to comprise at least one heterologous gene encoding growth hormone, prior to its being introduced into the embryo. Conventionally, growth hormone is exogenously administered to patients with diseases in which growth hormone is deficient. The aspect of the invention provides a suitable alternative. The heterologous gene encoding growth hormone, supplies the source of growth hormone, thereby obviating the need for exogenous administration. This may be accomplished by transfecting a somatic cell with an expression vector including the gene encoding growth hormone. The method of such a genetic manipulation is similar to the method described above, and is well known in the art. Further, the DNA sequences encoding growth hormone can be obtained from natural sources, for example, a normal organism or tissue sample, or can be synthetically produced using sequences obtained from the literature or from publicly accessible databases. These methods and resources are widely employed and known in the art.

Additionally, the invention provides a method of producing an autologous stem cell, by providing a somatic cell, which has been manipulated genetically to comprise at least one heterologous gene encoding phenylalanine hydroxylase (PAH), prior to its being introduced into the embryo. Mutations may arise in the PAH locus resulting in PAH deficiency. Depending on the nature of the mutation, different effects on the breakdown of phenylalanine may result. If PAH is not produced or if it is mutated, phenylketoneuria (PKU), non-PKU-hyperphenylalaninemia (non-PKU HPA), and variant PKU may result. Individuals with these conditions have heightened levels of phenylalanine in their blood plasma, since it can not be broken down in the absence of PAH. PKU, if left untreated, results in irreversible mental retardation due to lack of cognitive development. Studies show that PKU sufferers have lower levels of the neurotransmitter, dopamine, which may contribute to mental retardation (Denecke J, et al., J. Inherit. Metab. Dis. (2000) 23(8):849-851).

Current treatment for PKU includes limiting or restricting the ingestion of dietary phenylalanine thereby precluding the accumulation of dangerously high plasma concentrations of phenylalanine. Additionally, PKU is currently treated with BH₄, a cofactor in the breakdown of phenylalanine, which has been shown to reduce plasma levels of phenylalanine. The aspect of the invention provides a superior alternative or supplement to the currently available treatments of dietary restriction and BH₄. In an embodiment, a somatic cell is genetically manipulated to comprise, a heterologous gene encoding PAH prior to its being introduced into the embryo. The heterologous gene encoding PAH supplies a source of PAH to patients with PAH deficiency. The DNA sequences encoding the sequences for PAH can be obtained from natural sources, for example a normal organism or tissue sample, or can be synthetically produced using sequences obtained from the literature or from publicly accessible databases. These methods and resources are widely employed and known in the art.

Another aspect of the invention provides a method of producing an autologous stem cell by providing a somatic cell, which has been manipulated genetically to comprise at least one heterologous gene that complements a deficiency of the somatic cell, for example sickle cell disease, cystic fibrosis, phenylketonuria, thalassemia, Tay Sachs disease, Fanconi anemia, Hartnup disease, pyruvate dehydrogenase deficiency, congenital fructose intolerance (aldolase B deficiency), or galactosemia. All of these diseases are known to frequently arise from defects or mutations in a single individual gene and can be considered to arise from a deficiency in a cell. Accordingly, this aspect of the invention provides that a somatic cell can be manipulated genetically to comprise at least one heterologous gene that complements a deficiency of the somatic cell for any of these diseases and disorders. In so doing, the aspect of the invention may help to ameliorate these diseases and disorders by expression of a heterologous gene that complements the deficiency. By way of example, an expression vector may be synthesized containing all that is necessary to produce a normal version of a defective gene product at high levels when transfected into a cell. The methods of such a genetic manipulation are similar to those methods described above and are well known in the art. Further, the DNA sequences encoding hexosaminidase A, or other gene sequences implicated in any of the above mentioned diseases, can be obtained from natural sources, for example, a normal organism or tissue sample, or can be synthetically produced using sequences obtained from the literature or from publicly accessible databases. These methods and resources are widely employed and known in the art.

Sickle cell disease is an autosomal recessive disease common in areas where malaria is endemic. It is caused by a point mutation in the hemoglobin locus resulting in a valine rather than a glutamic acid as the amino acid at position six. This altered hemoglobin crystallizes readily at low oxygen tension. Erythrocytes from individuals who are homozygous for this mutation change from the typical discoid shape to a sickle shape. As a consequence, these sickle shaped erythrocytes become trapped in capillaries or are damaged in transport, resulting in anemia. In its heterozygous form, the disadvantages associated with sickle shaped erythrocytes are balanced by an increased resistance to Plasmodium falciparum malaria, likely because parasitized cells tend to sickle and are removed from circulation. Therefore, the sickle cell genotype in heterozygous form confers resistance to malaria.

Cystic fibrosis is an autosomal recessive disease that stems from a defect in the gene for the cystic fibrosis transmembrane conductance regulator (CFTCR) protein. CFTCR is a transmembrane protein that functions as a selection transporter. Defects in the CFTCR locus result in a decrease in fluid and salt secretion that can result in conduit obstruction, such as the blockage of exocrine outflow from the pancreas, the accumulation of dehydrated mucus in the airways, and obstruction of the intestinal passageway (meconium ileus), lacrimal passageway (high sweat electrolyte content), and pulmonary passageway (chronic bronchopulmonary infection and/or emphysema).

Thalassemia is a genetic form of anemia wherein affected individuals fail to properly synthesize hemoglobin, resulting in the production of small, pale, short-lived erythrocytes. Hemoglobin is comprised of four polypeptides—two alpha chains and two beta chains. Defects in either chain can result in thalassemia. Alpha thalassemia arises from a gene deletion resulting in a reduction in the synthesis of alpha chain. Beta thalassemia is caused by point mutations in the beta chain locus and is subdivided into two categories according to pathogenesis. Beta thalassemia major patients are homozygous for the defective genes; symptoms include slow growth, jaundice, enlarged heart, liver, and spleen, and thin bones. Beta thalassemia minor patients are heterozygous for the defective gene and suffer a milder form of anemia.

Tay Sachs disease is a fatal autosomal recessive disease in which harmful quantities of ganglioside GM2 accumulate in nerve cells of the central nervous system. Oneset is typically during infancy, but a rare adult-onset version has been observed. Tay Sachs disease is caused by insufficient activity of hexosaminidase A, which is responsible for catalyzing ganglioside degradation.

Fanconi anemia (FA) is an fatal autosomal recessive disease characterized by anemia and bone marrow failure. At least eight genes contribute to FA; products of five of these genes have been reported to form a nuclear complex, leading to the ubiquitination of a FA protein (D2), which may be involved in DNA damage response mechanisms. The most common cause of death in FA patients is bone marrow failure, followed in frequency by leukemia and solid tumors.

Hartnup's disease is characterized by a pellagra-like photosensitive rash, cerebellar ataxia, emotional instability, and aminoaciduria (Baron D N, et al., Lancet (1956) 271(6940):421-428). The disease presents with kidney and intestine defects, ataxia, personality changes, migraine headaches, and photophobia. It is caused by defective amino acid transport which leads to excessive loss of monoamino monocarboxylic acids in the urine and poor gastrointestinal absorption. (Scriver C R, N. Engl. .J Med. (1965) 273: 530-532).

Pyruvate dehydrogenase complex deficiency (PDCD) is a common neurodegenerative disorder and is linked to abnormal mitochondrial metabolism. It arises from a malfunction of the citric acid cycle, a major biochemical process that derives energy from carbohydrates, thus depriving the body of energy. Consequently, lactate builds up abnormally, which manifests in nonspecific symptoms, for example lethargy, poor feeding, and tachypnea. Progressive neurological symptoms may include developmental delay, intermittent ataxia, poor muscle tone, abnormal eye movements, and seizures. The pyruvate dehydrogenase complex is an enzymatic complex that converts pyruvate to acetyl CoA, one of two necessary substrates required to produce citrate. A deficiency in this complex limits the production of citrate, the first substrate in the citric acid cycle. Accordingly, the cycle cannot proceed and alternative metabolic pathways are stimulated in an attempt to override the defect and to produce acetyl CoA. However, an energy deficit remains, particularly in the central nervous system. The most common form of PDCD is caused by mutations in the X-linked E1 alpha gene. Other forms have been attributed to alterations in recessive genes.

Congenital fructose intolerance is an autosomal recessive form of carbohydrate intolerance due to aldolase B deficiency. Typically, onset is in infancy; the disease is characterized by hypoglycemia, with variable manifestations of fructosuria, fructosemia, anorexia, vomiting, failure to thrive, jaundice, splenomegaly, and an aversion to foods containing fructose. Mutational and structural analysis of the aldolase B gene has suggested that the integrity of the quaternary structure of aldolase B is involved in maintaining its full catalytic function (Rellos P, et al., J. Biol. Chem. (2000) 275(2): 1145-1151).

“Galactosemia” is the failure of the body to metabolize galactose, resulting in the aberrant accumulation of galactose 1-phosphate, causing damage to the liver, central nervous system, and various other body systems. Galactosemia is an autosomal recessive disorder. At least three forms have been described: galactose-1 phosphate uridyl transferase deficiency, galactose kinase deficiency, and galactose-6-phosphate epimerase deficiency. Each results in the failure to break down galactose, resulting in accrual of upon ingestion of galactose derivatives upon ingestion, which may lead to intolerance to feeding, jaundice, vomiting, lethargy, irritability, convulsions, cirrhosis of the liver, cataract formation in the eye, and mental retardation.

### Method of Reprogramming a Somatic Cell

This aspect of the invention provides a method of reprogramming a somatic cell by providing a somatic cell of a first subject, introducing it to a recipient embryo of a second subject, allowing the chimeric embryo to develop, and selecting an embryonic stem cell that is derived from the somatic cell.

Traditionally, stem cell researchers believed that only early embryonic stem cells had the potential to become any type of cell in the body, and that once stem cells had been localized to a specific organ, they could only differentiate into cells specific to that organ. Recent research, however, indicates that adult stem cells may be less specialized than scientists initially thought. Adult stem cells that would once have been assumed to be committed to becoming specific mature cells can be reprogrammed to mature into an entirely different cell line. One study reported that adult hematopoietic stem cells gave rise not only to bone marrow and hematopoietic cells as expected, but also to lung, digestive system, liver, and skin cells. (Krause D S, et al., Cell (2001) 105(3):369-377). Similarly, studies have reported that muscle stem cells could give rise to new hematopoietic stem cells, and further, that these hematopoietic stem cells can also revert back to producing muscle cells. (Thomson J A, et al., Science (1998) 282(5391):1145-1147). Therefore, these studies have revealed a broader potential for adult stem cells to reprogram, thereby giving rise to a greater multiplicity of cell types than originally believed.

Moreover, recent studies have shown that pluripotency can be acquired. In somatic cell nuclear transfer, the nuclear content of a terminally differentiated, somatic cell can reprogram and acquire the potential to differentiate, like an embryonic stem cell (McGrath J, et al., Science (1983) 229:1300-1302). This technique can produce embryonic cells that give rise to an entire organism (Campbell K H S, et al., Nature (1996) 39:64-66; Wilmut I, et al., Nature (1997) 385:810-813). In this technique, the nuclear content of an oocyte is replaced by the nuclear content of a somatic cell. This may be accomplished by merging the somatic cell and the enucleated oocyte, for example, by either fusion or injection. In the fusion method, a somatic cell is placed in contact with an enucleated oocyte. An electrical pulse is applied to the two cells, causing the somatic cell's nucleus to enter the enucleated oocyte. In the injection method, the nuclear content of the somatic cell is directly microinjected into the enucleated oocyte. In these studies, the nucleus of the somatic cell provides the genetic information, while the oocyte provides relevant nutrients and other energy-producing materials. The cell then develops in an embryonic environment and reprograms to acquire the ability to be pluripotent. The cell develops into a blastocyst, at which point, the pluripotent stem cells may be isolated from the ICM. These pluripotent stem cells have the ability to differentiate into any cell type and can support full development (Wakayama T, et al., Science (2001) 292(5517):740-743; Wakayama T, et al., Nature (1998) 394(6691):369-374). Other studies report the reprogramming of specific nuclear activities in cloned animals, such as genome-wide gene expression patterns, DNA methylation, genetic imprinting, histone modifications, and telomere length regulation, illustrating the complexity of reprogramming (Tamada H and Kikyo N, Cytogenet. Genome Res. (2004); 105(2-4):285-291). Collectively, these studies demonstrate that the nuclear material of a somatic cell, including its nuclear genome, is capable of reprogramming to exhibit pluripotent activity when placed in an enucleated oocyte and allowed to develop.

One aspect of the invention discloses a method of reprogramming that does not involve nuclear transfer. Instead, the invention provides a method of reprogramming a somatic cell by introducing a somatic cell to a recipient embryo, allowing a chimeric embryo to develop, and selecting an embryonic stem cell that is derived from the somatic cell. Thus, the invention provides a novel method of reprogramming a somatic cell that introduces a somatic cell into an embryo, without nuclear transfer. Accordingly, this aspect of the invention provides that the whole somatic cell, not merely its nuclear content, reprograms. Stated another way, the nuclear content reprograms within the entirety of the somatic cell.

Another aspect of the invention provides a method of reprogramming a somatic cell by providing a somatic cell of a first subject, introducing it to a recipient embryo of a second subject at or near the eight cell stage, allowing the chimeric embryo to develop, and selecting an embryonic stem cell that is derived from the somatic cell. In an embodiment, both the donor somatic cell and recipient embryonic cell are derived from the same origin. An in vivo example of an application of this method is where an inbred mouse, which is commercially available, provides the somatic cell, for example fibroblasts from the tail, which is then introduced to the recipient embryo of an inbred mouse of the identical strain, i.e., is syngeneic. In this example, the invention provides for the introduction of two different, but syngeneic, cell types.

A further aspect of the invention also relates to the composition of an autologous stem cell produced by either or both of two methods, mentioned previously. First, a method of producing an autologous stem cell for a donor subject by providing a somatic cell from a donor, introducing it into a recipient embryo, allowing the chimeric embryo to develop, and selecting an autologous embryonic stem cell that has developed from the somatic cell. Second, a method of reprogramming a somatic cell by providing a somatic cell of a first subject, introducing it to a recipient embryo of a second subject, allowing the chimeric embryo to develop, and selecting an embryonic stem cell that is derived from the somatic cell. This aspect of the invention relates to the composition of the product, which is an autologous stem cell that is produced by either or both of two methods. The first method provides the method of producing an autologous stem cell, and this aspect of the invention provides the composition of the autologous stem cell. The second method provides a method of reprogramming a somatic cell. An autologous stem cell may be produced by the method of reprogramming a somatic cell, and this aspect of the invention relates to that composition by either or both of these two methods.

The invention yet further provides the progeny of an autologous stem cell produced by either or both of these two methods. Like the parent stem cell, these progeny also possess the ability to differentiate and to self-renew. It is this ability to self-renew that allows stem cells to maintain themselves throughout the lifetime of an organism. This aspect of the invention thus relates to an autologous stem cell, as well as any and all of its progeny, when it is produced by either or both of the two methods generally described previously.

In an embodiment, the invention provides one or more differentiated cell derived from an autologous stem cell produced by either or both of the two methods described above.

### INDUSTRIAL APPLICABILITY

The invention provides methods of producing autologous stem cells and reprogramming somatic cells that are generally useful in the study, prevention, and treatment of a wide variety of disease states.

1. A method of producing an autologous embryonic stem cell for a donor subject comprising:
(a) providing a somatic cell of a donor subject;
(b) introducing the somatic cell into an embryo of a recipient subject to produce a chimeric embryo;
(c) allowing the chimeric embryo to develop; and
(d) selecting a developed autologous embryonic stem cell from the somatic cell.

2. The method of claim 1, wherein the somatic cell is genetically distinguishable from the recipient cell.

3. The method of claim 2, wherein the somatic cell comprises a detectable marker.

4. The method of claim 3, wherein the detectable marker comprises a reporter gene.

5. The method of claim 4, wherein the reporter gene is selected from luciferase, green fluorescent protein, and beta-galactosidase.

6. The method of claim 1, wherein the somatic cell is an adult stem cell.

7. The method of claim 6, wherein the adult stem cell is selected from a mesenchymal stem cell, a hematopoietic stem cell, and a neural stem cell.

8. The method of claim 1, wherein the somatic cell is introduced into the embryo at or near the eight cell stage.

9. The method of claim 1 or claim 8, wherein the autologous embryonic stem cell is a blastocyst.

10. The method of claim 1, wherein the somatic cell has been manipulated genetically, prior to its introduction into the embryo.

11. The method of claim 10, wherein the somatic cell is manipulated to comprise at least one heterologous gene.

12. The method of claim 11, wherein the heterologous gene complements a deficiency of the somatic cell.

13. The method of claim 11, wherein the heterologous gene enhances at least one function or activity of the somatic cell.

14. The method of claim 11, wherein the heterologous gene encodes telomerase reverse transcriptase.

15. The method of claim 14, wherein the telomerase reverse transcriptase is human telomerase transcriptase.

16. The method of claim 12, wherein the deficiency is a chromosomal deficiency.

17. The method of claim 16, wherein the chromosomal deficiency is recessive.

18. The method of claim 11, wherein the heterologous gene encodes growth hormone or phenylalanine hydroxylase.

19. The method of claim 12, wherein the heterologous gene complements a disorder selected from sickle cell disease, cystic fibrosis, phenylketonuria, thalassemia, Tay Sachs disease, Fanconi's anemia, Hartnup disease, pyruvate dehydrogenase deficiency, congenital fructose intolerance, and galactosemia.

20. A method of reprogramming a somatic cell comprising:
(a) providing a somatic cell of a first subject;
(b) providing an embryo of a second subject;
(c) introducing the somatic cell into the embryo to produce a chimeric embryo;
(d) allowing the embryo to develop further; and
(e) selecting an embryonic stem cell that is derived from the somatic cell.

21. The method of claim 20, wherein the somatic cell is introduced into the embryo at or near the eight cell stage.

22. The method of claim 20, wherein the first subject is the same as the second subject.

23. An autologous stem cell produced by the method of claim 1 or claim 20.

24. A progeny of the autologous stem cell of claim 1 or claim 20.

25. A differentiated cell derived from the autologous stem cell of claim 1 or claim 23.
